SEQUENCE LISTING (1 sequence)

SEQ ID NO: 1 - protein, 708 aa, unidentified
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAVEGPSATGPEIIRSPQDDASSPHLQVMLQIHLPGRHTLFVRAMIDSGASGNFIDHEYVAQNGIPLRIKDWPILVEAIDGRPIASGPVVHETHDLIVDLGDHREVLSFDVTQSPFFPVVLGVRWLSTHDPNITWSTRSIVFDSEYCRYHCRMYSPIPPSLPPPAPQPPLYYPVDGYRVYQPVRYYYVQNVYTPVDEHVYPDHRLVDPHIEMIPGAHSIPSGHVYSLSEPEMAALRDFVARNVKDGLITPTIAPNGAQVLQVKRGWKLQVSYDCRAPNNFTIQNQYPRLSIPNLEDQAHLATYTEFVPQIPGYQTYPTYAAYPTYPVGFAWYPVGRDGQGRSLYVPVMITWNPHWYRQPPVPQYPPPQPPPPPPPPPPPPSYSTL

(19) United States
(12) Patent Application Publication
(10) Pub. No.: US 2025/0114336 A1
(43) Pub. Date: Apr. 10, 2025

(54) USE OF ANTI-RETROVIRAL PROTEASE INHIBITORS FOR THE TREATMENT OF AMYOTROPHIC LATERAL SCLEROSIS (ALS)

(71) Applicant: The Regents of the University of Colorado, a body corporate, Denver, CO (US)

(72) Inventor: Alexandra Whiteley, Boulder, CO (US)

(21) Appl. No.: 18/909,779

(22) Filed: Oct. 8, 2024

Related U.S. Application Data
(60) Provisional application No. 63/543,408, filed on Oct. 10, 2023.

(51) Int. Cl. A61K 31/427

(52) U.S. Cl. CPC A61K 31/427

(57) ABSTRACT

The present invention is directed to a method for inhibiting the self-cleavage of PEG10 for treatment of ALS comprising the step of administering to a subject a therapeutically effective amount of ritonavir.

### CROSS-REFERENCE TO RELATED APPLICATIONS

This U.S. Non-Provisional Application claims the benefit of and priority to U.S. Provisional Application No. 63/543,408, filed Oct. 10, 2023. The entire specification, claims, and figures of the above-referenced application is hereby incorporated, in its entirety by reference.

### SEQUENCE LISTING

The instant application contains contents of the electronic sequence listing (Corrected-90245.01111-Sequence Listing.xml; Size: 2,710 bytes; and Date of Creation: Oct. 31, 2024) is herein incorporated by reference in its entirety.

### TECHNICAL FIELD

The present invention relates to methods and compositions for the treatment of amyotrophic lateral sclerosis (ALS) or Angelman syndrome (AS), specifically the novel use of the anti-retroviral protease inhibitor ritonavir to decrease PEG10 in a target cell, as well as inhibit self-cleavage of PEG10.

### BACKGROUND

ALS is a fatal neuromuscular disorder characterized by progressive paralysis that affects upwards of 30,000 Americans. It is a devastating disease that afflicts people, primarily in the fifth to sixth decade of life, and typically kills within 3-5 years of diagnosis. There is no cure for ALS, and there are no known biomarkers for the disease and few treatment options—none of which significantly prolong lifespan. Two leading therapeutics for ALS are Riluzole and Edaravone have shown some limited therapeutic success. Both work by inhibiting excitotoxic glutaminergic signaling and oxidative stress, respectively. However, these drugs have shown limited benefit in reducing symptoms and do not significantly lengthen lifespan. Clearly, there is a significant unmet medical need for novel ALS therapeutics.

Historically, neurodegenerative diseases involving protein buildup have been challenging to target. There are multiple, high-profile examples of biologic therapies (such as the monoclonal antibodies gantenerumab and solanezumab) intended to facilitate degradation of protein aggregates in Alzheimer's which have been successful to varying degrees at clearing the aggregates, but having not demonstrated major improvement in symptoms or disease progression. Other ALS drugs that work downstream of protein aggregation have significant side effects due to the fact that they alter generalized neuronal function. For example, Riluzole inhibits excitotoxicity, which is thought to contribute to the symptoms of ALS due to neuronal dysfunction. However, Riluzole is often poorly tolerated, resulting in elevated liver enzymes and rarely can cause serious interstitial lung disease, especially in people of Japanese descent.

One of the major roadblocks to developing ALS therapies is that very little is known about the molecular mechanism of disease. In general, ALS shares a few key molecular perturbations. ALS patient tissues exhibit protein accumulation and aggregation, which is thought to contribute to neuronal dysfunction. However, the specifics of how aggregates lead to neuronal dysfunction are poorly understood. There are multiple genes that have been identified as risk factors for a familial form of ALS (fALS), which accounts for approximately 10% of total ALS cases; the other cases are considered sporadic (sALS), with no known genetic etiology. While there are multiple unique genetic mutations that can lead to fALS, each genetic mutation is unique, current treatment options therapeutically target downstream effects that are shared amongst familial and sporadic ALS. As such, there exists a long-felt need for an effective treatment of ALS.

### SUMMARY OF THE INVENTION

In another aspect, the present invention includes methods and compositions for decreasing PEG10 levels in a target cell, as well as inhibiting the self-cleavage of PEG10.

In a preferred aspect, the invention includes the novel use of the anti-retroviral protease inhibitor ritonavir to decrease the level of PEG10 in a target cell, preferably in a target human cell.

In another aspect, the present invention includes the novel use of anti-retroviral protease inhibitors to treat a disease or condition related to elevated levels of PEG, such as ALS or AS. Specifically, in a preferred aspect the invention includes the novel use of the anti-retroviral protease inhibitor ritonavir to decrease the level of PEG10 in a target cell, preferably in a human cell as a treatment for ALS or AS.

In another preferred aspect, the invention includes the novel use of the anti-retroviral protease inhibitor ritonavir to inhibit self-cleavage of PEG10 preferably in a human cell. In another aspect, the present invention includes the novel use of anti-retroviral protease inhibitors to treat ALS. Specifically, in a preferred aspect the invention includes the novel use of the anti-retroviral protease inhibitor ritonavir to inhibit self-cleavage of PEG10, preferably in a human cell as a treatment for ALS or AS.

Additional aspects of the invention will be apparent by the specification, claims, and figures provided herein.

### BRIEF DESCRIPTION OF THE FIGURES

FIG. 1: Western blot of PEG10 self-cleavage testing with antiretroviral compounds. HEK293 cells were transfected with either WT or a cleavage-incompetent mutant form of human PEG10 and incubated for 24 hours with the indicated concentrations of drug. After 24 hours, cells were lysed and imaged by western blot.

FIG. 2A-D: PEG10 abundance is decreased by ritonavir, but not indinavir. a-d) Bands identified in FIG. 1 were quantified and normalized to Tubulin for a) gag-pol, b) gag, c) CA fragment, and d) CA^{NTD} fragment. Shown is mean+/−SEM for four representative experiments; one sample from untreated indinavir and 50 uM of indinavir and ritonavir were not collected. Statistics were measured by One-way ANOVA with multiple comparisons to untreated; statistically significant comparisons are shown with asterisks. ANOVA summary significance is shown with straight line.

FIG. 3A-C: Self-cleavage of PEG10 is modestly affected by ritonavir. a) ratio of PEG10 gag-pol to gag for quantitation in FIG. 2. b) ratio of CA fragment to gag, and c) CA^{NTD} fragment to gag. Shown is mean+/−SEM for four representative experiments; one sample from untreated indinavir and 50 uM of indinavir and ritonavir were not collected. Statistics were measured by One-way ANOVA with multiple comparisons to untreated; statistically significant comparisons are shown with asterisks. ANOVA summary significance is shown with straight line.

### DETAILED DESCRIPTION OF THE INVENTION

In one aspect, the present invention includes methods and compositions for inhibiting the self-cleavage of PEG10, preferably in a human cell. In on preferred embodiment, the present invention includes the novel use of anti-retroviral protease inhibitors to inhibit PEG10 levels in a cell, as well as to inhibit self-cleavage of PEG10. In a preferred aspect, the present invention includes the novel use of anti-retroviral protease inhibitors to decrease levels of PEG10 (SEQ ID NO. 1) in a target cell, preferably in a human cell. In another preferred embodiment, present invention includes the novel use of the anti-retroviral protease inhibitor ritonavir to inhibit self-cleavage of PEG10 in a target cell, preferably in a human cell. Ritonavir is represented structurally as Formula (I) below.

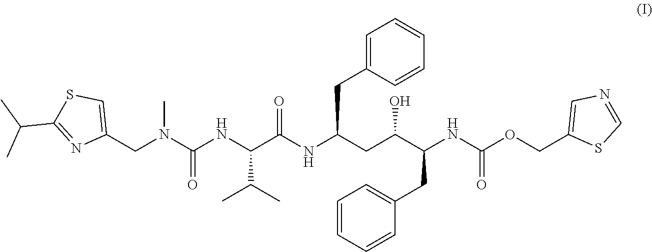 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

Ritonavir is also known to be an HIV protease inhibitor and an inhibitor of cytochrome P450 monooxygenase, especially, CYP3A4 as disclosed in U.S. Pat. No. 6,037,157. Ritonavir can be prepared using any of the procedures disclosed in U.S. Pat. Nos. 5,142,056, 5,151,438, 5,354,866, 5,362,912, 5,491,253, 5,508,409, 5,526,677, 5,539,122, 5,541,206, 5,541,328, 5,541,334, 5,543,549, 5,543,551, 5,543,552, 5,545,750, 5,552,558, 5,554,783, 5,565,418, 5,565,604, 5,567,823, 5,569,777, 5,580,984, 5,583,232, 5,583,233, 5,591,860, 5,597,926, 5,597,927, 5,597,928, 5,608,072, 5,616,714, 5,616,720, 5,616,776, 5,625,072, 5,625,092, 5,635,523, 5,648,497, 5,654,466, 5,659,044, 5,659,045, 5,670,675, 5,674,882, 5,679,797, 5,696,270, 5,786,500, 5,837,873, 5,846,987, 5,886,036, 5,892,052, 6,017,928, 6,150,530 and 6,531,610 which are incorporated in their entirety herein by reference.

Exemplary ritonavir formulations are disclosed in U.S. Pat. Nos. 5,142,056, 5,151,438, 5,354,866, 5,362,912, 5,484,801, 5,491,253, 5,508,409, 5,526,677, 5,539,122, 5,541,206, 5,541,328, 5,541,334, 5,543,549, 5,543,551, 5;543,552, 5,545,750, 5,552,558, 5,554,783, 5,559,158, 5,565,418, 5,565,604, 5,567,823, 5,569,777, 5,580,984, 5,583,232, 5,583,233, 5,591,860, 5,597,926, 5,597,927, 5,597,928, 5,608,072, 5,610,193, 5,616,714, 5,616,720, 5,616,776, 5,625,072, 5,625,092, 5,635,523, 5,648,497, 5,654,466, 5,659,044, 5,659,045, 5,670,675, 5,674,882, 5,679,797, 5,696,270, 5,725,878, 5,786,500, 5,837,873, 5,846,987, 5,876,749, 5,886,036, 5,892,052, 5,948,436, 6,017,928, 6,150,530, 6,232,333, 6,458,818, 6,521,651, and 6,531,610, published U.S. Patent Application No. US-0032619-A1 and published PCT Application Nos. WO 93/23361, WO 94/14436, WO 95/07696, WO 95/09614, WO 95/20384, WO 98/22106, WO 00/74677, WO 01/52821 and WO 02/096395 which are incorporated in their entirety herein by reference. It should be noted that while the aforementioned references disclose various ritonavir formulations as examples, the present invention is not limited thereto.

In another aspect, the present invention includes the novel use of anti-retroviral protease inhibitors to treat a condition related to, or cause by elevated levels of PEG, such as ALS or AS. In a preferred aspect, the present invention includes the novel use of anti-retroviral protease inhibitors to decrease levels of PEG10 (SEQ ID NO. 1) in a cell, and preferably a target cell in a human subject suffering from, or at risk of developing ALS or AS.

In another preferred embodiment, present invention includes the novel use of the anti-retroviral protease inhibitor ritonavir to inhibit self-cleavage of PEG10, preferably in a human cell, and preferably a target cell in a human subject suffering from, or at risk of developing ALS or AS.

In another aspect, the present invention includes methods of treating a disease of condition that is caused, at least in part, by elevated levels of PEG10, such as ALS or AS in a subject in need thereof, comprising the step of administering a therapeutically effective amount of a PEG10 inhibitor, which in a preferred embodiment is ritonavir. In another aspect, the present invention includes methods of treating a disease of condition that is caused, at least in part, by elevated levels of PEG10, such as ALS or AS in a subject in need thereof, comprising the step of administering to a subject in need thereof a pharmaceutical compositions including a therapeutically effective amount of PEG10 inhibitor and a pharmaceutically acceptable carrier.

In another preferred embodiment, the present invention includes a method of treating ALS or AS by reducing the level of PEG10 in a target cell(s) of a subject, the method comprising administering a therapeutically effective amount of ritonavir to a subject in need thereof.

In another preferred embodiment, the present invention includes a method of treating ALS or AS by reducing the level of PEG10 in a target cell(s) of a subject, the method comprising administering to a subject in need thereof a pharmaceutical compositions including a therapeutically effective amount of ritonavir and a pharmaceutically acceptable carrier.

In another preferred embodiment, the present invention includes a method of treating ALS or AS by inhibiting the self-cleavage of PEG10, the method comprising administering a therapeutically effective amount of ritonavir to a subject in need thereof.

In a preferred embodiment, the present invention includes a method of treating ALS or AS by inhibiting the self-cleavage of PEG10, the method comprising administering to a subject in need thereof a pharmaceutical compositions including a therapeutically effective amount of ritonavir and a pharmaceutically acceptable carrier.

While it is possible for the active compound, namely ritonavir to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary, shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrants will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet. Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet. Other conventional ingredients include antioxidants, colorants, flavoring agents, preservatives and taste-masking agents. Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated. The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety. Solid formulations for oral administration may be formulated to be immediate and/or modified release. Modified release formulations include delayed-, sustained-, pulsed-, controlled-, targeted and programmed release. Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

Formulations suitable for topical administration (e.g., transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichorotetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams, or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 g to about 250 mg per kilogram body weight of the subject per day. Where the active compound is a salt, an ester, prodrug, or the like, the amount administered is calculated on the basis of the parent compound and so the actual weight to be used is increased proportionately.

### Definitions

“Amyotrophic lateral sclerosis” or “ALS” are terms understood in the art and are used herein to denote a progressive neurodegenerative disease that affects upper motor neurons (motor neurons in the brain) and/or lower motor neurons (motor neurons in the spinal cord) and results in motor neuron death. As used herein, the term “ALS” includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

“Angelman syndrome” (AS) is a neurodevelopmental disorder characterized by severe developmental delay or intellectual disability, severe speech impairment, gait ataxia and/or tremulousness of the limbs, seizures, microcephaly and a unique behavior with an inappropriate happy demeanor that includes frequent laughing, affinity for water, smiling, and excitability. Microcephaly and seizures are also common. Developmental delays are first noted at around age six months; however, the unique clinical features of Angelman syndrome do not become manifest until after age one year, and it can take several years before the correct clinical diagnosis is obvious.

As used herein “PEG10 levels” or “PEG10 expression” means amount of PEG10 protein, or discrete gag or pol subunits present in a cell (e.g., a biological sample or a control sample). As used herein “PEG10 protein” means a protein that is substantially identical to all or a part of SEQ ID NO. 1, or its discrete gag or pol subunits or any protein having between 80-99% sequence homology with PEG10. Notably, disclosure of a nucleotide sequence further encompasses and incorporates by reference the corresponding amino acid sequence thereof. Similarly, disclosure of an amino acid sequence further encompasses and incorporates by reference the corresponding nucleotide sequence thereof.

As used herein, “inhibits,” “inhibition” or “decease” refers to the decrease in PEG10 levels relative to the normal wild type levels in a target cell. Inhibition may result in a decrease in PEG10 levels by less than 10%, 10%, 15%, 20%, 25%, 30%, 35%, 40%, 45%, 50%, 55%, 60%, 65%, 70%, 75%, 80%, 85%, 90%, 95% or 100%. In a specific embodiment, “inhibits,” “inhibition” or “decrease” refers to the decrease in the self-cleavage of PEG10 relative to the normal wild type level. Inhibition may result in a decrease in the self-cleavage of PEG10, by less than 10%, 10%, 15%, 20%, 25%, 30%, 35%, 40%, 45%, 50%, 55%, 60%, 65%, 70%, 75%, 80%, 85%, 90%, 95% or 100%.

By “treating” a disease, disorder, or condition is meant delaying an initial or subsequent occurrence of a disease, disorder, or condition; increasing the disease-free survival time between the disappearance of a condition and its reoccurrence; stabilizing or reducing one or more (e.g., two, three, four, or five) adverse symptom(s) associated with a condition; or inhibiting, slowing, or stabilizing the progression of a condition. The term “treating” also includes reducing (e.g., by at least 5%, 10%, 15%, 20%, 25%, 30%, 35%, 40%, 45%, 50%, 55%, 60%, 65%, 70%, 75%, 80%, 85%, 90%, or 95% the severity or duration of one or more (e.g., one, two, three, four, or five) symptoms of a disease (e.g., ALS) in a patient. Desirably, at least 20%, 40%, 60%, 80%, 90%, or 95% of the treated subjects have a complete remission in which all evidence of the disease disappears. In another desirable embodiment, the length of time a patient survives after being diagnosed with a condition and treated using the methods of the invention is at least 20%, 40%, 60%, 80%, 100%, 200%, or even 500% greater than (i) the average amount of time an untreated patient survives or (ii) the average amount of time a patient treated with another therapy survives.

As used herein, the term “prevent” or “preventing” refers to precluding, averting, obviating, forestalling, stopping, or hindering something from happening, especially by advance action. In an aspect, prevent or preventing refers to the ameliorating of one or more signs and symptoms associated with ALS or FTD. It is understood that where reduce, inhibit or prevent are used herein, unless specifically indicated otherwise, the use of the other two words is also expressly disclosed.

A “therapeutically effective amount” of a compound of a pharmaceutical composition thereof is an amount sufficient to provide a therapeutic benefit in the treatment of a disease or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent. A “therapeutically effective amount” may also mean “prophylactically effective amount” of a compound of the present invention is an amount sufficient to prevent a disease or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

Administration of a treatment may be effected by any method that enables delivery of the compositions to the site of action. These methods include oral routes, intraduodenal routes, parenteral injection (including intravenous, subcutaneous, intramuscular, intravascular or infusion), topical, and rectal administration. Dosage regimens may be adjusted to provide the optimum desired response. For example, a single bolus may be administered, several divided doses may be administered overtime, or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the composition and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The term “subject” refers to any animal. In certain embodiments, the subject is a mammal. In certain embodiments, the subject is a human (e.g., a man, a woman, or a child). The human may be of either sex, or may be at any stage of development. In certain embodiments, the subject has been diagnosed with ALS. In other embodiments, the subject is at risk of developing the neurodegenerative condition or disease, such as ALS. In certain embodiments, the subject is an experimental animal (e.g., mouse, rat, rabbit, dog, pig, or primate). The experimental animal may be genetically engineered.

As used herein, the phrase “in need thereof” means that the animal or mammal has been identified as having a need for the particular method or treatment. In some embodiments, the identification can be by any means of diagnosis. In any of the methods and treatments described herein, the animal or mammal may be in need thereof. In some embodiments, the animal or mammal is in an environment or will be traveling to an environment in which a particular disease, disorder, or condition is prevalent.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The invention is further described with reference to the following non-limiting Examples.

### EXAMPLES

### Example 1: Small-Molecule Screen for PEG10 Inhibitor

The PEG10 protease domain resembles retrotransposon and retroviral proteases and is predicted to share a similar structure. Applicants and others have shown that PEG10 self-cleaves, and that this self-cleavage generates a fragment which when overexpressed changes gene expression in the cell and may contribute to human disease. Many small molecule inhibitors of retroviral proteases exist and are very effective at inhibiting self-cleavage (often referred to as ‘self-processing’). Applicants sought to identify a small molecule that similarly inhibits self-cleavage of PEG10.

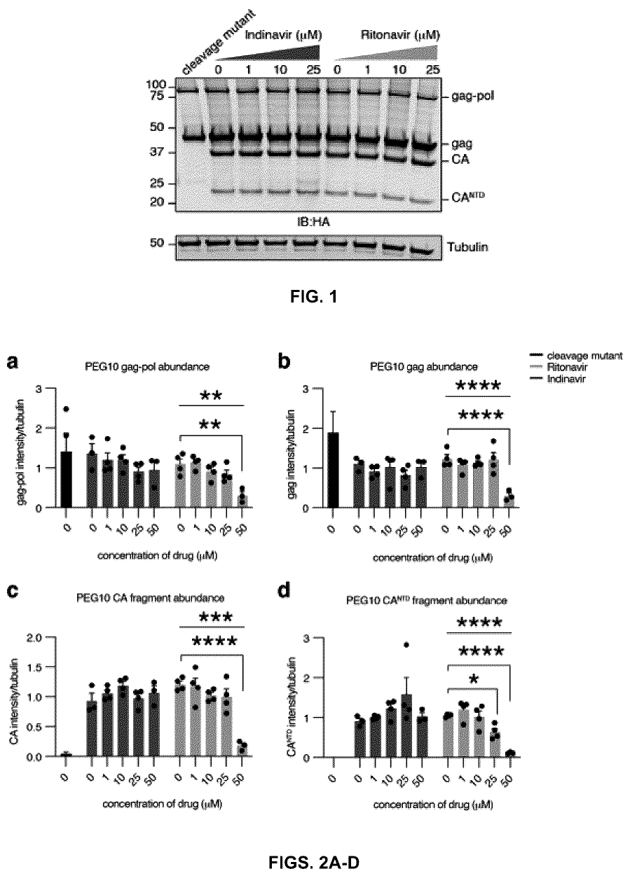

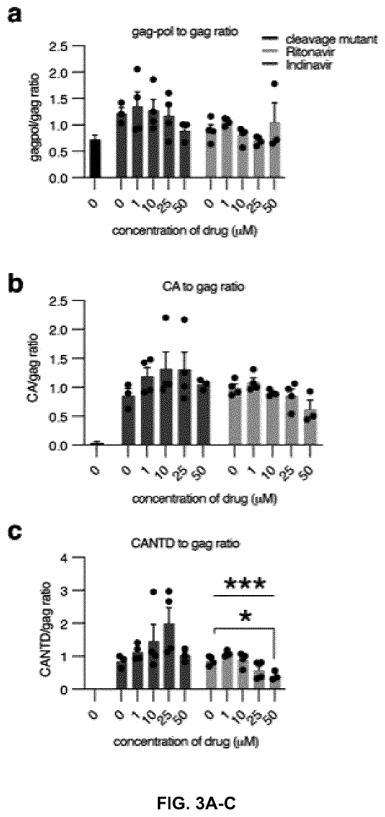

Applicants performed an assay using western blot to assess self-cleavage of transfected PEG10 (FIG. 1) with a negative control where the PEG10 protease is mutated. Increasing doses of indinavir or ritonavir were added to the cells for 24 hours before lysis for western blotting. Applicants found Applicants ritonavir uniquely decreases PEG10 expression in the cell (FIG. 2) compared to indinavir. We also found that there was a modest effect on self-cleavage of PEG10 (FIG. 3c). As such, ritonavir could be useful in ALS or Angelman's syndrome, where PEG10 abundance is high and could contribute to disease. It could also be useful as an abortifacient, as the PEG10 protease is essential for murine fetal development.

### REFERENCES

1. Golda, M., Mótyán, J. A., Mahdi, M., and Tözsér, J. (2020). Functional Study of the Retrotransposon-Derived Human PEG10 Protease. Int. J. Mol. Sci. 21, 2424. 10.3390/ijms21072424.
2. Clark, M. B., Janicke, M., Gottesbuhren, U., Kleffmann, T., Legge, M., Poole, E. S., and Tate, W. P. (2007). Mammalian Gene PEG10 Expresses Two Reading Frames by High Efficiency—1 Frameshifting in Embryonic-associated Tissues. J. Biol. Chem. 282, 37359-37369. 10.1074/jbc.M705676200.
3. Black, H. H., Hanson, J. L., Roberts, J. E., Leslie, S. N., Campodonico, W., Ebmeier, C. C., Holling, G. A., Tay, J. W., Matthews, A. M., Ung, E., et al. (2023). UBQLN2 restrains the domesticated retrotransposon PEG10 to maintain neuronal health in ALS. eLife 12, e79452. 10.7554/eLife.79452.
4. Wang, Y., Lv, Z., and Chu, Y. (2015). HIV protease inhibitors: a review of molecular selectivity and toxicity. HIVAIDS—Res. Palliat. Care, 95. 10.2147/HIV.S79956.
5. Pandya, N.J., Wang, C., Costa, V., Lopatta, P., Meier, S., Zampeta, F. I., Punt, A. M., Mientjes, E., Grossen, P., Distler, T., et al. (2021). Secreted retrovirus-like GAG-domain-containing protein PEG10 is regulated by UBE3A and is involved in Angelman syndrome pathophysiology. Cell Rep. Med. 2, 100360. 10.1016/j.xcrm.2021.100360.
6. Shiura, H., Ono, R., Tachibana, S., Kohda, T., Kaneko-Ishino, T., and Ishino, F. (2021). PEG10 viral aspartic protease domain is essential for the maintenance of fetal capillary structure in the mouse placenta. Dev. Camb. Engl. 148, dev199564. 10.1242/dev.199564.

What is claimed is:

1. A method of treating amyotrophic lateral sclerosis (ALS) in a subject in need thereof, comprising the step of inhibiting the self-cleavage of PEG10, wherein said step of inhibiting comprises administering a therapeutically effective amount of ritonavir according to Formula (I): 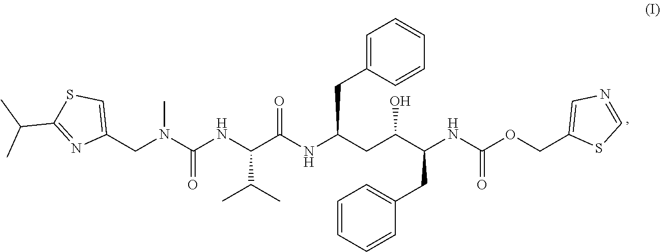 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]
or a pharmaceutically acceptable salt thereof.

2. The method of claim 1, wherein said PEG10 comprises the amino sequence according to SEQ ID NO. 1, or a sequence having at least 80% sequence homology with SEQ ID NO. 1.

3. The method of claim 1, wherein said subject comprises a human.

4. A method of treating amyotrophic lateral sclerosis (ALS) in a subject in need thereof, the method comprising administering a pharmaceutical composition having a therapeutically effective amount of ritonavir according to Formula (I): 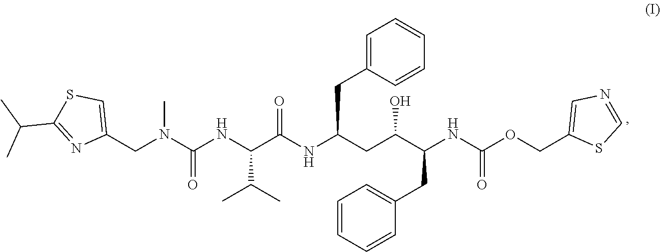 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]
or a pharmaceutically acceptable salt thereof, and a pharmaceutically acceptable carrier.

5. The method of claim 4, wherein said therapeutically effective amount of ritonavir decreases the level of PEG10 in a target cell of the subject.

6. The method of claim 4, wherein said therapeutically effective amount of ritonavir inhibits the self-cleavage of PEG10 in a target cell of the subject.

7. The method of claim 4, wherein said subject comprises a human.

8. A method of inhibiting PEG10 in a target cell of a subject in need thereof, comprising the step of administering to a subject a therapeutically effective amount of ritonavir.

9. The method of claim 8, wherein said subject comprises a human.

10. The method of claim 8, wherein said ritonavir comprises a compound according to Formula (I): 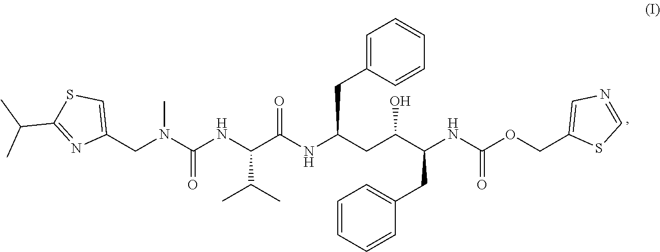 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]
or a pharmaceutically acceptable salt thereof.

11. The method of claim 8, wherein inhibiting PEG10 in a target cell of a subject treats ALS or Angelman syndrome (AS) in the subject.
